(19) United States
(12) Patent Application Publication
(10) Pub. No.: US 2005/0222184 A1
(43) Pub. Date: Oct. 6, 2005

(54) Process for preparing (s)-tetrahydro-a-(1-methylethyl)-2-oxo-1(2h)-pyrimidineacetic acid

(76) Inventors: Giorgio Bertolini, Sesto San Giovanni (Milano) (IT); Luca Feliciotti, Sesto San Giovanni (Milano) (IT); Marco Frigerio, Milano (IT); Massimo Losa, Pogliano Milanes (Milano) (IT)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/515,964

(22) PCT Filed: May 28, 2003
(86) PCT No.: PCT/IB03/02262

§ 371 Date: Nov. 23, 2004

(30) Foreign Application Priority Data
May 30, 2002 (IT) MI2002A 001168

(51) Int. Cl. A61K 31/513; C07D 239/02

(52) U.S. Cl. 514/269; 544/318

(57) ABSTRACT

A process for preparing (S)-tetrahydro-a-(1-methylethyl)-2-oxo-1(2H)-pyrimidineacetic acid, an intermediate that is useful in the synthesis of HIV protease inhibitors such as, for example, those described in U.S. Pat. No. 5,914,332, is described. The process under consideration comprises the following steps:—L-valine is reacted with acrylonitrile;—the N-(2-cyanoethyl)-L-valine thus obtained is isolated and then reacted with an alkyl chloroformate;—the N-(2-cyanoethyl)-N-(alkoxycarbonyl)-L-valine thus obtained is hydro-genated in the presence of a hydrogenation catalyst, preferably rhodium;—the N-(3-aminopropyl)-N-(methoxycarbonyl)-L-valine thus obtained is cyclized to give the desired compound.

The present invention relates to a novel process for preparing intermediates that may be used for preparing compounds with antiviral activity, and in particular HIV protease inhibitors having the formula given below: 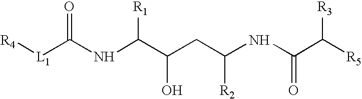
in which
R₁ and R₂ are independently selected from the group consisting of: lower alkyl, cycloalkylalkyl and arylalkyl;
R₃ is lower alkyl, hydroxyalkyl or cycloalkylalkyl;
R₄ is aryl;
R₅ is 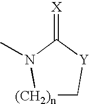
   in which n is 1, 2 or 3, X is O, S, or NH and Y is —O— or —N(R₆)— in which R₆ is hydrogen, lower alkyl, cycloalkyl, cycloalkylalkyl, aryl or arylalkyl; and
L₁ is —O—, —S—, —N(R₇)— in which R₇ is hydrogen, lower alkyl, cycloalkyl or cycloalkylalkyl, —O-alkylenyl, —S-alkylenyl, —S(O)-alkylenyl, —S(O)₂-alkylenyl, —N(R₇)-alkylenyl in which R₇ is defined as above, -alkylenyl-O—, -alkylenyl-S—, -alkylenyl-N(R₇)— in which R₇ is defined as above, alkylenyl, alkenylenyl; or a pharmaceutically acceptable salt, ester or prodrug thereof.

The preparation of such compounds is described, for example, in U.S. Pat. No. 5,914,332, which is incorporated herein by reference. Among these, the compound of main relevance is the compound known as Lopinavir, the structural formula of which is given below.

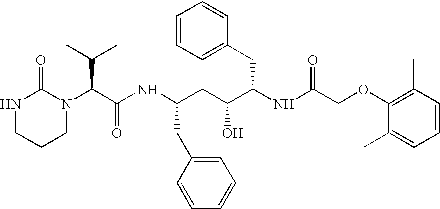

### PRIOR ART

The intermediate of interest is (S)-tetrahydro-a-(1-methylethyl)-2-oxo-1(2H)-pyrimidine-acetic acid, shown below, 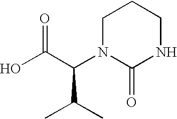
the preparation of which is also described in the abovementioned US patent.

In particular, in U.S. Pat. No. 5,914,332, (S)-tetrahydro-a-(1-methylethyl)-2-oxo-1(2H)-pyrimidine-acetic acid is obtained by reacting valine with acrylonitrile and methyl chloroformate, and then hydrogenating on Raney-nickel the product thus obtained, as shown by the reaction scheme below. 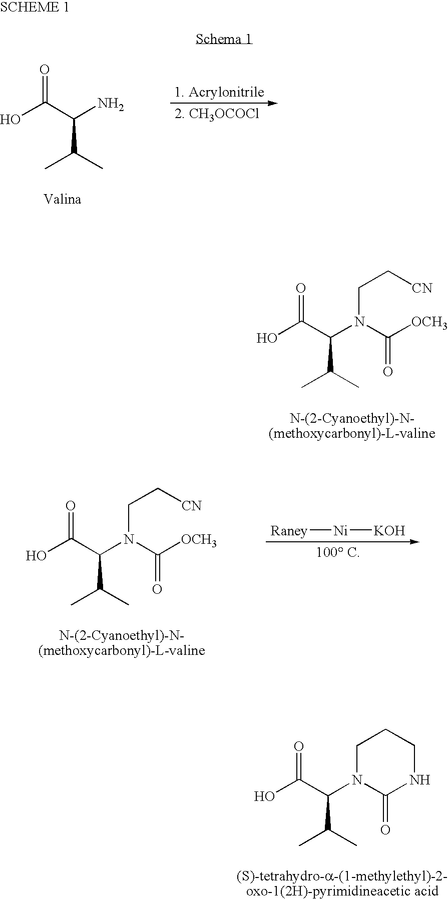
(S)-tetrahydro-α-(1-methylethyl)-2-oxo-1(2H)-pyrimidineacetic acid.

According to the process discussed above, the (S)-tetrahydro-α-(1-methylethyl)-2-oxo-1(2H)-pyrimidineacetic acid is obtained in an overall yield of 25%. In addition, apart from the rather modest yield, the process under consideration has a second non-negligible drawback, namely the use of a catalyst based on Raney-nickel. Specifically, as is known, nickel is a metal that is not disposed of easily; secondly, Raney-nickel may cause allergies and give rise to sensitization phenomena. In addition, Raney-nickel is classified as an agent that can cause irreversible effects and is thus considered potentially carcinogenic.

### DESCRIPTION OF THE INVENTION

A process for preparing (S)-tetrahydro-α-(1-methylethyl)-2-oxo-1(2H)-pyrimidineacetic acid has now been found, characterized not only by yields that are surprisingly superior to those of the process described in U.S. Pat. No. 5,914,332, but also, in its preferred embodiment, by the use of a catalyst that is less toxic and easier to dispose of than nickel, with obvious advantages in terms of the environment and health at work.

The process according to the present invention is characterized in that it comprises the following steps:
L-valine is reacted with acrylonitrile;
the N-(2-cyanoethyl)-L-valine thus obtained is isolated and then reacted with an alkyl chloroformate, preferably methyl chloroformate;
the N-(2-cyanoethyl)-N-(alkoxycarbonyl)-L-valine thus obtained is hydro-genated in the presence of a hydrogenation catalyst, preferably rhodium;
the N-(3-aminopropyl)-N-(methoxycarbonyl)-L-valine thus obtained is cyclized to give the desired compound.

In the optimum embodiment of the invention, step (a) is performed in water at a temperature of 0-25° C., and preferably at 0-5° C. In particular, the L-valine is reacted with approximately equimolar amounts of acrylonitrile; the reaction is preferably performed with 1-5 M concentrations of the two compounds.

For the purposes of the present invention, the expression “the N-(2-cyanoethyl)-L-valine thus obtained is isolated” means that the product obtained in step (a) is isolated from the reaction mixture in amorphous or crystalline form, in a purity at least greater than or equal to 95% and preferably 97%. The isolation under consideration may be performed by the usual methods that will be obvious to a person skilled in the art; the product will preferably be precipitated, filtered and dried under vacuum.

Step (b) is preferably performed in water, normally working at a pH of between 8.0 and 12.0 (preferably between 9.0 and 10.5) and at a temperature of between 0 and 40° C. and preferably between 20 and 25° C. In this case also, the N-(2-cyanoethyl)-L-valine is reacted with an excess of an alkyl chloroformate, preferably methyl chloroformate; the reaction is preferably performed with 0.5-3 M concentrations of the two compounds.

As mentioned previously, the hydrogenation catalyst referred to in step (c) is preferably rhodium and even more preferably rhodium supported on charcoal. The hydrogenation is performed at a pressure of 4-7 bar and preferably 6-7 bar and at a temperature of 35-65° C. and preferably 40-60° C., preferably working in basic medium in the presence of ammonia gas, ammonium hydroxide or sodium methoxide, preferably ammonia gas; the solvent used is usually an alkyl alcohol, preferably methanol or aqueous-alcoholic mixtures.

Finally, as regards the cyclization, this is preferably performed in water at the reflux temperature of the solvent, i.e. at about 100° C. This reaction is conveniently performed by basic catalysis; this cyclization is promoted by working at a pH of between 12 and 13; the pH is preferably regulated using NaOH.

To allow the process according to the present invention to be understood more clearly, it is given schematically below.

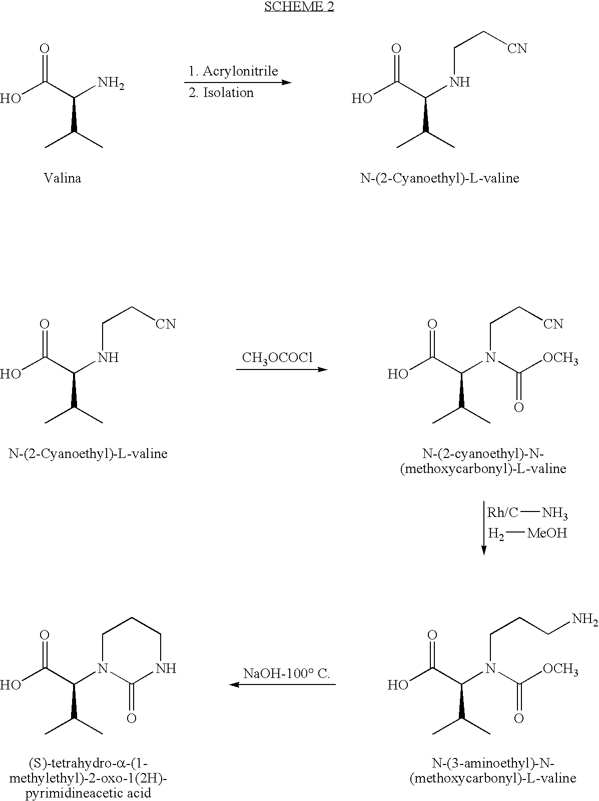

The (S)-tetrahydro-α-(1-methylethyl)-2-oxo-1(2H)pyrimidineacetic acid is then isolated according to standard methods that will be obvious to those skilled In the art; it is preferably extracted with methylene chloride after acidification of the reaction mixture, and then dried under vacuum.

As may be seen from the examples that follow, via the process of the present invention, the (S)-tetrahydro-α-(1-methylethyl)-2-oxo-1(2H)-pyrimidineacetic acid is obtained in an overall yield of 38%, i.e. in a yield 52% higher than that obtained by working according to the process described in U.S. Pat. No. 5,914,332. In addition, besides the advantages in terms of the environment and health at work discussed previously, the present process involves a hydrogenation at temperatures that are distinctly lower and safer (50° C.) than those used in U.S. Pat. No. 5,914,332 (100° C.) without, however, adversely affecting, but rather improving, the overall yield.

The examples that follow are given purely for the purpose of illustration and should not be understood as limiting the invention.

### EXAMPLE 1

### N-(2-Cyanoethyl)-L-valine

L-valine (100 g) was suspended in water (100 ml) and an 85% solution of potassium hydroxide (56 g) in water (100 ml) was added at 20° C. The reaction mixture was stirred at this temperature until the valine was fully dissolved.

The solution was cooled to 0-5° C. and acrylonitrile (45 g) was added slowly over about 30 minutes at 0-5° C. The reaction mixture was stirred at 0-5° C. for 4-5 hours. Water (250 ml) was added and the solution was acidified to pH 5 with concentrated hydrochloric acid (about 70 ml). The suspension was then stirred at 0-5° C. for 1 hour and the solid was filtered off and washed with water (25 ml). The solid was dried at 60° C. under vacuum to give 137 g of N-(2-cyanoethyl)-L-valine (91% yield). m.p. 245-250° C.; ¹³C-NMR (50 MHz, D₂O) δ: 171.76, 117.27, 68.34, 42.59, 28.86, 17.79, 16.91, 14.48; ¹H-NMR (200 MHz, D₂O) δ: 3.43 (d, 1H), 3.30 (t, 2H). 2.89 (t, 2H), 2.18-2.09 (m, 1H), 0.93 (d, 3H), 0.89 (d, 3H); IR (KBr) cm⁻¹: 3467, 2260, 1577; MS (El): 171 [M+1], 130, 125, 84, 81.

### EXAMPLE 2

### N-(2-Cyanoethyl)-N-(methoxycarbonyl)-L-valine

N-(2-cyanoethyl)-L-valine (120 g) was dissolved in a solution of sodium hydroxide pearls (22.6 g) in water (360 ml). The pH was adjusted to 9.5-10.5 with 30% sodium hydroxide (about 12 ml) and the reaction mixture was stirred until the N-(2-cyanoethyl)-L-valine was fully dissolved. Methyl chloroformate (100 g) was slowly added dropwise at 20-25° C., with simultaneous addition of 30% sodium hydroxide solution (about 144 ml), while maintaining the pH between 9.0 and 10.5. The reaction mixture was stirred at 20-25° C. for 20-30 minutes.

Further methyl chloroformate (33 g) and 30% sodium hydroxide (about 86 ml) were then simultaneously added dropwise at 20-25° C. while maintaining the pH between 9.0 and 10.5. The reaction mixture was stirred at 20-25° C. for 20-30 minutes.

Methylene chloride (240 ml) was added and the reaction mixture was acidified slowly at 20-25° C. with concentrated hydrochloric acid (about 168 ml) to pH 1.5. The phases were separated and the aqueous phase was extracted with methylene chloride (240 ml). The combined organic phases were evaporated under vacuum and the crude N-(2-cyanoethyl)-N-(methoxycarbonyl)-L-valine thus obtained was used directly for the following reaction without further purification.

### EXAMPLE 3

### N-(3-Aminopropyl)-N-(methoxycarbonyl)-L-valine

The crude N-(2-cyanoethyl)-N-(methoxycarbonyl)-L-valine obtained in Example 2 was dissolved in methanol (240 ml) and a solution of ammonia gas (72 g) in methanol (360 ml) was added, followed by addition of wet 5% rhodium-on-charcoal (2.4 g solids). The reaction mixture was then hydrogenated at 6-7 bar and 50° C. At the end of the reaction, the catalyst was filtered off and washed with methanol (50 ml). The methanolic solution was then evaporated under vacuum to give the N-(3-aminopropyl)-N-(methoxycarbonyl)-L-valine, which was used directly for the following reaction without further purification.

### EXAMPLE 4

### (S)-tetrahydro-α-(1-methylethyl)-2-oxo-1(2H)-pyrimidineacetic acid

The crude N-(3-aminopropyl)-N-(methoxycarbonyl)-L-valine obtained in Example 3 was dissolved in water (580 ml) and aqueous 30% sodium hydroxide solution (130 ml). The reaction mixture was refluxed until the cyclization was complete. The reaction mixture was then cooled to 15-20° C. and sodium chloride (82 g) and methylene chloride (500 ml) were added. The aqueous phase was acidified with concentrated hydrochloric acid (about 120 ml) at pH 1 and the phases were separated. The aqueous phase was extracted with methylene chloride (2×500 ml) and the combined organic phases were evaporated under vacuum. The residue was treated with hot ethyl acetate (400 ml), cooled to 0-5° C. and filtered, the solid being washed with ethyl acetate (about 48 ml) to give after drying 75 g of crude product.

The crude product was dissolved in hot isopropanol (175 ml) and hot ethyl acetate (690 ml) was then added. The suspension was cooled slowly to 0-5° C. and the solid was filtered off and washed with ethyl acetate (about 50 ml) to give after drying at 50-60° C. under vacuum 59 g of (S)-tetrahydro-α-(1-methylethyl)-2-oxo-1(2H)-pyrimidineacetic acid (42% yield over three steps). m.p. 176-177° C.; ¹³C-NMR (50 MHz, DMSO) δ: 173.47, 156.26, 62.63, 42.53, 27.44, 22.64, 20.60, 19.82; ¹H-NMR (200 MHz, DMSO) δ: 12.56 (s, 1H), 6.38 (s, 1H), 4.25 (d, 1H), 3.35-3.06 (m, 4H), 2.03-2.15 (1H), 1.83-1.71 (m, 1H), 0.92 (d, 3H), 0.81 (d, 3H); IR (KBr) cm⁻¹: 3307, 1695, 1613; MS (El): 202 [M+2], 200, 157, 155, 141, 113.

1. Process for preparing 2S-(1-tetrahydro-pyrimid-2-only)-3-methyl butanoic acid 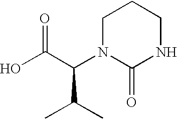
comprising the steps of:
a) reacting L-valine with acrylonitrile to form N-(2-cyanoethyl)-L-valine;
b) isolating the N-(2-cyanoethyl)-L-valine thus and then reacting with an alkyl chloroformate to form N-(2-cyanoethyl)-N-(alkoxycarbonyl)-L-valine;
c) hydrogenating the N-(2-cyanoethyl)-N-(alkoxycarbonyl)-L-valine in the presence of a hydrogenation catalyst to form N-(3-aminopropyl)-N-(methoxycarbonyl)-L-valine;
d) cyclizing the N-(3-aminopropyl)-N-(methoxycarbonyl)-L to form the 2S-(1-tetrahydro-pyrimid-2-only)-3-methyl butanoic acid.

2. Process according to claim 1, wherein the said alkyl chloroformate is methyl chloroformate.

3. Process according to claim 1, wherein step (a) is performed in water.

4. Process according to claim 1, wherein step (a) is performed at a temperature of 0-25° C.

5. Process according to claim 1, wherein the L-valine is reacted with approximately equimolar amounts of acrylonitrile.

6. Process according to claim 1, wherein the isolating step of step b) further comprises filtering the N-(2-cyanoethyl)-L-valine and drying the N-(2-cyanoethyl)-L-valine under vacuum.

7. Process according to claim 1, wherein the reacting step of step (b) is performed in water.

8. Process according to claim 1, wherein the reacting step of step (b) is performed at a pH of between 8.0 and 12.0.

9. Process according to claim 1, wherein the reacting step of step (b) is performed at a temperature of between 0 and 40° C.

10. Process according to claim 1, wherein the hydrogenation catalyst is rhodium.

11. Process according to claim 1, wherein step (c) is performed at a pressure of 4-7 bar and at a temperature of 35-65° C.

12. Process according to claim 1, wherein step (c) is performed by working in basic medium in the presence of ammonia gas, ammonium hydroxide or sodium methoxide.

13. Process according to claim 1, wherein the hydrogenation referred to in step (c) is performed by working in the presence of ammonia gas.

14. Process according to claim 1, wherein step (c) is performed in an alkyl alcohol.

15. Process according to claim 1, wherein step (d) is performed in water.

16. Process according to claim 15, wherein step (d) is performed by basic catalysis.

17. Process according to claim 16, wherein step (d) is performed at a pH of between 12 and 13 and at the reflux temperature of water.

18. Process for preparing an HIV protease inhibitor having the formula: 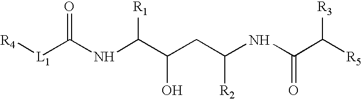
wherein
R₁ and R₂ are independently selected from the group consisting of: lower alkyl, cycloalkylalkyl and arylalkyl;
R₃ is lower alkyl, hydroxyalkyl or cycloalkylalkyl;
R₄ is aryl;
R₅ is 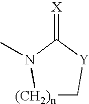
in which n is 1, 2 or 3, X is O, S, or NH and Y is —O— or —N(R₆)— in which R₆ is hydrogen, lower alkyl, cycloalkyl, cycloalkylalkyl, aryl or arylalkyl; and
L₁ is —O—, —S—, —N(R₇)— in which R₇ is hydrogen, lower alkyl, cycloalkyl, cycloalkylalkyl, —O-alkylenyl, —S-alkylenyl, —S(O)-alkylenyl, —S(O)₂-alkylenyl, —N(R₇)-alkylenyl in which R₇ is defined as above, -alkylenyl-O—, -alkylenyl-S—, -alkylenyl-N(R₇)— in which R₇ is defined as above, alkyleny, alkenylenyl;
or a pharmaceutically acceptable salt, ester or prodrug thereof, comprising the step of using 2S-(1-tetrahydro-pyrimid-2-only)-3-methyl butanoic acid made in accordance with the process of claim 1 as an intermediate in the synthesis of the HIV protease inhibitor.

19. Process for preparing Lopinavir having the formula: 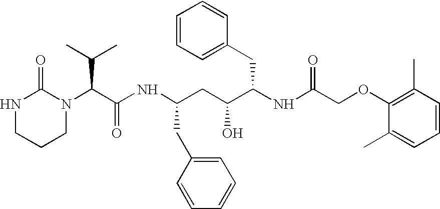
comprising the step of using 2S-(1-tetrahydro-pyrimid-2-only)-3-methyl butanoic acid made in accordance with the process of claim 1 as an intermediate in the synthesis of the Lopinavir.

20. Process according to claim 1, wherein step (a) is performed at a temperature of 0-5°.

21. Process according to claim 1, wherein the reacting step of step (b) is performed at a pH of between 9.0 and 10.5.

22. Process according to claim 1, wherein the reacting step of step (b) is performed at a temperature of between 20 and 25° C.

23. Process according to claim 10, wherein the rhodium is supported on charcoal.

24. Process according to claim 11, wherein the pressure is from 6-7 bar.

25. Process according to claim 11, wherein the temperature is between 40-60° C.

26. Process according to claim 14, wherein the alkyl alcohol is methanol.

27. Process according to claim 1, wherein step (c) is performed in an aqueous alcoholic mixture.

28. Process according to claim 14, wherein the alkyl alcohol is methanol.

29. 2S-(1-tetrahydro-pyrimid-2-only)-3-methyl butanoic acid made in accordance with the process of claim 1.

30. An HIV protease inhibitor made in accordance with the process of claim 18.

31. Lopinavir made in accordance with the process of claim 19.
